(19) United States
(12) Patent Application Publication
(10) Pub. No.: US 2011/0160600 A1
(43) Pub. Date: Jun. 30, 2011

(54) ERECTILE FUNCTION INDEX MEASURING AND ANALYZING SYSTEM AND MEASURING AND ANALYZING METHOD THEREOF

(76) Inventors: Hsien-Tsai WU, Shoufong (TW); Chun-Ho Lee, Jhuangwei Township (TW); Chih-Jung Chew, Hualien City (TW)

(21) Appl. No.: 12/975,877

(22) Filed: Dec. 22, 2010

(30) Foreign Application Priority Data
Dec. 29, 2009 (TW) 098145513

(51) Int. Cl. A61B 5/02

(52) U.S. Cl. 600/500

(57) ABSTRACT

An erectile function index measuring and analyzing system includes a measuring device and an analyzing device. The measuring device is configured to output a standard pulse signal based upon a penile artery pulse of a subject that is measured while a penis of the subject is in a relaxed state, a first comparative pulse signal based upon a penile artery pulse that is measured after releasing the penis from external pressure larger than a systolic pressure, and a second comparative pulse signal based upon a penile artery pulse that is measured while the penis is in an erect state. The measuring device is further configured to measure heartbeat for outputting an electrocardiogram signal. According to the above signals, the analyzing device is operable to obtain a vasodilatation index, a pulse wave velocity and an erectile index to serve as erectile function indices used for determining erectile function of the subject.

### CROSS-REFERENCE TO RELATED APPLICATION

This application claims priority of Taiwanese Application No. 098145513, filed on Dec. 29, 2009.

### BACKGROUND OF THE INVENTION

1. Field of the Invention

The present invention relates to a system and method for measuring and analyzing a physiology index, and a measuring and analyzing method thereof, more particularly to an erectile function index measuring and analyzing system, and a measuring and analyzing method thereof.

2. Description of the Related Art

When a male is under a sexual stimulus, the corpus cavernosum nerve of the autonomic nervous system of the male acts to generate nitric oxide (NO), a gaseous neurotransmitter responsible for signal transmission in living organisms. After being generated by endothelial cells, nitric oxide is diffused to vascular smooth muscles to dilate the vascular smooth muscles to thereby increase an amount of blood flow in a penis of the male. Nitric oxide further relaxes penile smooth muscles so as to advance tumescence and hyperemia of corpus cavernosum. Then, the penis of the male is in an erect state naturally. A normal erection is attributed to action of the blood vessels and the nervous system. Insufficient rigidity of a penis in the erect state is known as impotence, and called as erectile dysfunction in clinical medicine.

According to experience in clinical medicine, the causes of erectile dysfunction generally include psychogenic causes, organic causes, and medication. Erectile dysfunction attributed to psychogenic causes is mostly caused by psychological factors that result in inability of the central nervous system to stimulate erection. Erectile dysfunction attributed to organic causes is mostly caused by injury or abnormality of blood vessels, nerves, the endocrine system, or the corpus cavernosum. Long-term ingestion of hypotensors, antidepressants, tranquilizers, etc. also causes erectile dysfunction.

Although there are many factors that will affect erectile function, it has been found through researches in urological clinical medicine that erectile dysfunction is mostly attributed to organic causes. The most common organic cause is cardiovascular diseases, such as atherosclerosis. Currently, many researches have found that endothelial cell dysfunction is an initial symptom of atherosclerosis. Moreover, previous researches have found that there exists a significant relation between the function of penile endothelial cells and the erectile function. Therefore, screening the function of the penile artery in the long term will significantly advance early diagnosis of erectile dysfunction, and prevention of cardiovascular diseases in an early stage.

Current means for measuring and analyzing erectile dysfunction in clinical diagnosis include RigiScan, Penile Brachial Index (PBI), Doppler ultrasound, penile angiography, and so on.

Among the above-mentioned means, RigiScan is an instrument used mainly for screening a degree of tumescence and rigidity of an erect penis, and duration of erection. Results from RigiScan are generally acknowledged as a prime standard for diagnosis of erectile dysfunction. However, this instrument is expensive, and it is inconvenient in that a subject must be sleeping when performing measurement and analysis for determining the kind of erectile dysfunction is. PBI is a measurement that compares blood pressure in a penile artery with blood pressure in a brachial artery. Although it is relatively easy and convenient to perform PBI measurement, results therefrom are unstable since an environment and a mood of the subject easily affect reaction of the penile artery. Doppler ultrasound is used for measuring a condition of penile blood vessels and dynamic variation of blood flow in the penile blood vessels under erection, and results therefrom are extremely useful for clinical medicine. However, an ultrasound instrument is heavy and expensive, and the results may have an error attributed to human operation. The penile angiography is an invasive measuring method using X-ray for detection. Moreover, an instrument for the penile angiography is expensive and requires professional operation.

The above-mentioned instruments are either expensive or require professional operation, and the results therefrom may be unstable or have errors. Therefore, these disadvantages usually result in difficulty in diagnosis of erectile dysfunction.

### SUMMARY OF TEE INVENTION

Therefore, an object of the present invention is to provide an erectile function index measuring and analyzing system that is non-invasive, that is relatively low cost, and that is easy to operate.

Accordingly, an erectile function index measuring and analyzing system of the present invention comprises a measuring device and an analyzing device coupled to the measuring device.

The measuring device includes a pressure sensor that is configured to output a standard pulse signal based upon a penile artery pulse of a subject that is measured while a penis of the subject is in a relaxed state. The pressure sensor is further configured to output at least one of a first comparative pulse signal based upon a penile artery pulse of the subject that is measured after releasing the penis of the subject from external pressure larger than a systolic pressure of the subject, and a second comparative pulse signal based upon a penile artery pulse of the subject that is measured while the penis of the subject is in an erect state.

The analyzing device includes a signal processing unit, a digitizing unit coupled to the signal processing unit, and an analyzing unit coupled to the digitizing unit. The signal processing unit is configured to amplify and perform filter processing upon the standard pulse signal and said at least one of the first and second comparative pulse signals outputted by the pressure sensor of the measuring device to obtain a processed standard pulse signal and at least one of first and second processed comparative pulse signals. The digitizing unit is configured to digitize the processed standard pulse signal and said at least one of the first and second processed comparative pulse signals to obtain a digital standard pulse signal and at least one of a first digital comparative pulse signal and a second digital comparative pulse signal. The analyzing unit is configured to analyze signal parameters of the digital standard pulse signal and said at least one of the first and second digital comparative pulse signals, and to obtain at least one of a vasodilatation index with reference to the first digital comparative pulse signal and an erectile index with reference to the second digital comparative pulse signal to serve as an erectile function index used for determining erectile function of the subject.

According to another aspect of this invention, an erectile function index measuring and analyzing system comprises a measuring device and an analyzing device coupled to the measuring device.

The measuring device includes a pressure sensor, an electrode set for application to a hand and a foot of a subject, and an electrocardiograph coupled to the electrode set. The pressure sensor is configured to output a standard pulse signal based upon a penile artery pulse of the subject that is measured while a penis of the subject is in a relaxed state. The electrocardiograph is configured to measure heartbeat of the subject and output an electrocardiogram signal concurrent with measuring of the penile artery pulse of the subject.

The analyzing device includes a signal processing unit, a digitizing unit coupled to the signal processing unit, and an analyzing unit coupled to the digitizing unit. The signal processing unit is configured to amplify and perform filter processing upon the standard pulse signal and the electrocardiogram signal outputted by the measuring device to obtain a processed standard pulse signal and a processed electrocardiogram signal. The digitizing unit is configured to digitize the processed standard pulse signal and the processed electrocardiogram signal to obtain a digital standard pulse signal and a digital electrocardiogram signal. The analyzing unit is configured to analyze the digital standard pulse signal and the digital electrocardiogram signal, and to obtain a pulse wave velocity to serve as an erectile function index used for determining erectile function of the subject according to a predetermined travel distance and a time interval between the digital standard pulse signal and the digital electrocardiogram signal.

Another object of the present invention is to provide an erectile function index measuring and analyzing method that is capable of correctly screening a function of a penile artery, and that can advance early diagnosis of erectile dysfunction.

Accordingly, an erectile function index measuring and analyzing method of the present invention comprises the steps of:

a) measuring a penile artery pulse of a subject to obtain a standard pulse signal while a penis of the subject is in a relaxed state;

b) obtaining at least one of a first comparative pulse signal based upon a penile artery pulse of the subject that is measured after releasing the penis of the subject from external pressure larger than a systolic pressure of the subject, and a second comparative pulse signal based upon a penile artery pulse of the subject that is measured while the penis of the subject is in an erect state;

c) processing the standard pulse signal and said at least one of the first and second comparative pulse signals to obtain a digital standard pulse signal and at least one of a first digital comparative pulse signal and a second digital comparative pulse signal, respectively; and

d) according to signal parameters of the digital standard pulse signal and said at least one of the first and second digital comparative pulse signals, obtaining at least one of a vasodilatation index with reference to the first digital comparative pulse signal and an erectile index with reference to the second digital comparative pulse signal to serve as an erectile function index used for determining erectile function of the subject.

According to another aspect of this invention, an erectile function index measuring and analyzing method comprises the steps of:

i) while a penis of a subject is in a relaxed state, measuring heartbeat of the subject to obtain an electrocardiogram signal and measuring a penile artery pulse of the subject to obtain a standard pulse signal;

ii) processing the electrocardiogram signal and the standard pulse signal to obtain a digital electrocardiogram signal and a digital standard pulse signal; and

iii) according to a predetermined travel distance and a time interval between the digital electrocardiogram signal and the digital standard pulse signal, obtaining a pulse wave velocity to serve as an erectile function index used for determining erectile function of the subject.

### BRIEF DESCRIPTION OF THE DRAWINGS

Other features and advantages of the present invention will become apparent in the following detailed description of the preferred embodiments with reference to the accompanying drawings, of which:

FIG. 1 is a block diagram of a preferred embodiment of an erectile function index measuring and analyzing system of the present invention;

FIG. 2 is a flow chart illustrating a first preferred embodiment of an erectile function index measuring and analyzing method of the present invention;

FIG. 3 is a flow chart illustrating steps of the erectile function index measuring and analyzing method of the first preferred embodiment for obtaining a pulse wave velocity;

FIG. 4 shows a digital standard pulse signal that is obtained by processing a standard pulse signal based upon a penile artery pulse of the subject that is measured while the penis of the subject is in the relaxed state;

FIG. 5 shows a first digital comparative pulse signal that is obtained by processing a first comparative pulse signal based upon a penile artery pulse of the subject that is measured after releasing the penis of the subject from the external pressure;

FIG. 6 is a schematic diagram illustrating increasing and decreasing slopes obtained by analyzing connection between peaks of waveforms using Hilbert-Huang transformation;

FIG. 7 is a schematic diagram illustrating a time interval between the digital electrocardiogram signal and the digital standard pulse signal;

FIG. 8 is a flow chart illustrating a second preferred embodiment of an erectile function index measuring and analyzing method of the present invention; and

FIG. 9 shows an erectile index obtained using the erectile function index measuring and analyzing method of the second preferred embodiment.

### DETAILED DESCRIPTION OF THE PREFERRED EMBODIMENTS

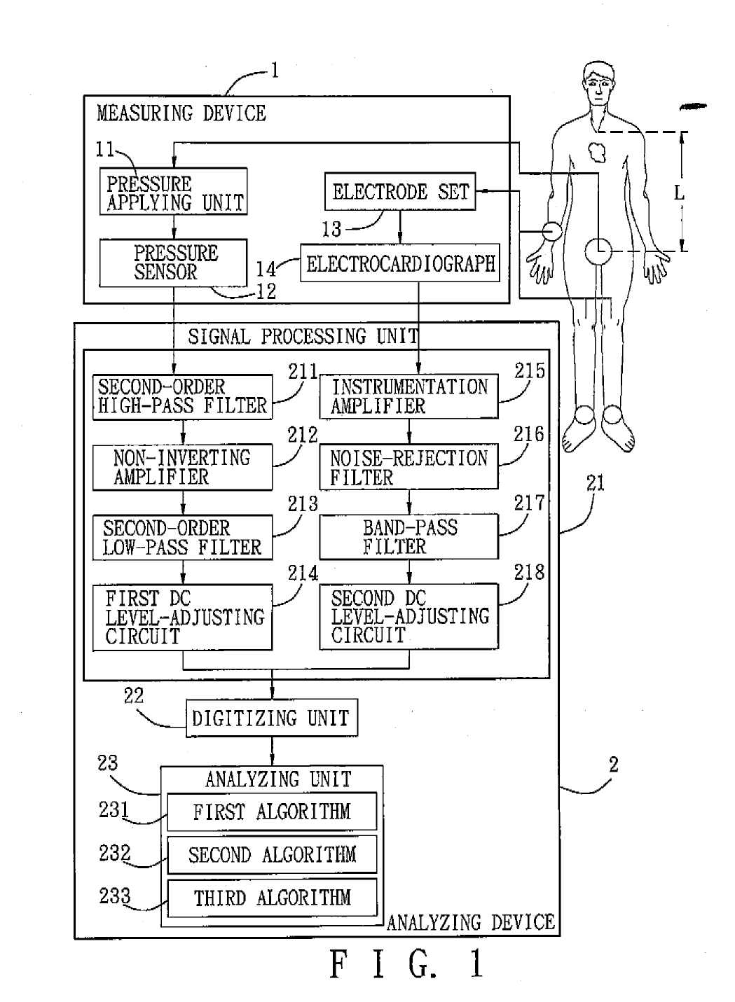

Referring to FIG. 1, the preferred embodiment of an erectile function index measuring and analyzing system of this invention includes a measuring device 1 and an analyzing device 2 coupled to the measuring device 1.

The measuring device 1 includes a pressure applying unit 11, a pressure sensor 12 coupled to the pressure applying unit 11, an electrode set 13 for application to a hand and a foot of a subject, and an electrocardiograph 14 coupled to the electrode set 13. The pressure applying unit 11 is operable in a pressure-applying mode, where the pressure applying unit 11 is configured to apply external pressure to a penis of the subject, or a pressure-releasing mode, where the pressure applying unit 11 is configured to release the penis of the subject from the external pressure. Preferably, the external pressure is larger than a systolic pressure of the subject. In this embodiment, the pressure applying unit 11 is a pressure cuff, and is configured to surround the penis of the subject. Preferably, the pressure applying unit 11 is configured to surround a penile shaft of the penis of the subject. The pressure sensor 12 is associated operatively with the pressure applying unit 11 for measuring a penile artery pulse of the subject to output a pulse signal based upon a penile artery pulse of the subject. In this embodiment, the pressure sensor 12 is in fluid communication with the pressure cuff (the pressure applying unit 11) without coming into contact with penile skin of the subject to thereby measure the penile artery pulse of the subject. In other embodiments, the pressure sensor 12 can be a skin-contact-type pressure sensor, such as a clamping-type pressure sensor, a probe-type pressure sensor and a sticker-pad-type pressure sensor, or any other types of pressure sensors that are well known to those skilled in the art. The electrocardiograph 14 is configured to measure heartbeat of the subject and output an electrocardiogram signal in a conventional manner.

The analyzing device 2 includes a signal processing unit 21, a digitizing unit 22 coupled to the signal processing unit 21, and an analyzing unit 23 coupled to the digitizing unit 22. The signal processing unit 21 is configured to amplify and perform filter processing upon the pulse signal and the electrocardiogram signal outputted by the measuring device 1 to obtain a processed pulse signal and a processed electrocardiogram signal. The digitizing unit 22 is configured to digitize the processed pulse signal and the processed electrocardiogram signal to obtain a digital pulse signal and a digital electrocardiogram signal. The analyzing unit 23 is configured to analyze the digital pulse signal and the digital electrocardiogram signal, and to perform at least one of first, second and third algorithms 231-233 for obtaining at least one of a vasodilatation index, a pulse wave velocity (PWV) and an erectile index to serve as an erectile function index used for determining erectile function of the subject.

For processing the pulse signal from the measuring device 1, the signal processing unit 21 includes a second-order high-pass filter 211, a non-inverting amplifier 212, a second-order low-pass filter 213, and a first DC level-adjusting circuit 214. For processing the electrocardiogram signal from the measuring device 1, the signal processing unit 21 further includes an instrumentation amplifier 215, a noise-rejection filter 216, a band-pass filter 217, and a second DC level-adjusting circuit 218. In this embodiment, the band-pass filter 217 includes a pair of high-pass filters and a low-pass filter, and a bandwidth thereof is in a range from 0.98 Hz to 19.4 Hz.

The first preferred embodiment of an erectile function index measuring and analyzing method performed using the system illustrated in FIG. 1 will now be described in the succeeding paragraphs.

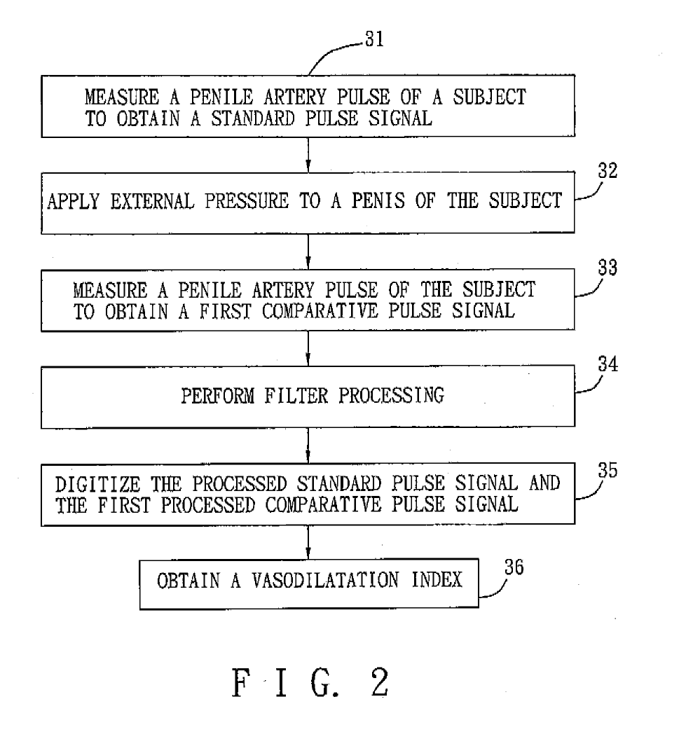

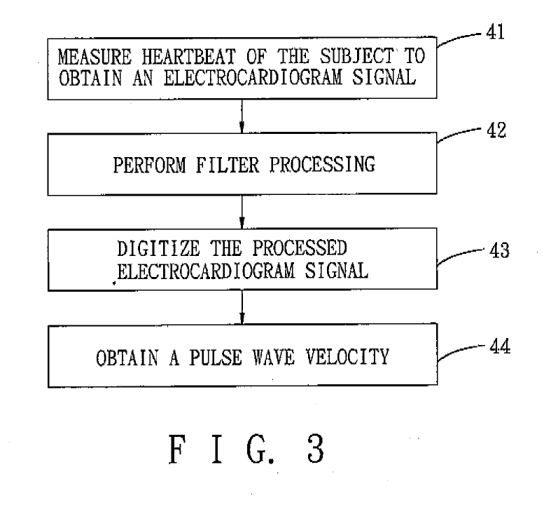

Referring to FIGS. 1 to 3, before the pressure applying unit 11 applies the external pressure to the penis of the subject, the penis of the subject is in a relaxed state for a duration of time. At this time, the pressure sensor 12 is associated operatively with the pressure applying unit 11 to measure a penile artery pulse of the subject so as to obtain a standard pulse signal in step 31. Preferably, in the relaxed state, the penis is subjected to a constant pressure of 80 mmHg for 4 minutes before measuring the penile artery pulse. Then, the pressure sensor 12 is operable to measure the penile artery pulse of the subject for 2 minutes to obtain the standard pulse signal.

In step 32, the pressure applying unit 11 is operated in the pressure-applying mode to apply the external pressure larger than the systolic pressure of the subject to the penis of the subject for a duration of time. Then, the pressure applying unit 11 is operated in the pressure-releasing mode to release the penis of the subject from the external pressure. After releasing the penis of the subject from the external pressure, the pressure sensor 12 measures a penile artery pulse of the subject to obtain a first comparative pulse signal in step 33.

Preferably, the pressure applying unit 11 applies 300-mmHg external pressure to the penis of the subject for 2 minutes in the pressure-applying mode. When applying the 300-mmHg external pressure to the penis of the subject, a penile artery of the subject is completely blocked, such that blood cannot flow to the penis of the subject. Therefore, a pulse signal measured at this time is approximately a straight line. During this duration, endothelial cells are stimulated to liberate nitric oxide for controlling dilatation of blood vessels. After releasing the penis of the subject from the external pressure, the penis of the subject is under a reactive hyperemia period such that the endothelial cells continuously act so as to result in continuous dilatation of the blood vessels. Therefore, a continuous pulse signal after the straight line can be measured at this time.

In step 34, the signal processing unit 21 is operable to perform filter processing upon the standard pulse signal and the first comparative pulse signal to obtain a processed standard pulse signal and a first processed comparative pulse signal. It should be noted that bandwidth of the signals from the pressure sensor 12 is approximately 0-40 Hz, and therefore it is difficult to distinguish the signals from surrounding noise, particularly from 60-Hz interference noise. Accordingly, the second-order high-pass filter 211 is operable so as to remove DC signal components from the standard pulse signal and the first comparative pulse signal, followed by operation of the non-inverting amplifier 212 for gain amplification. Then, the second-order low-pass filter 213 is operable so as to remove noise attributed to rubbing of the pressure applying unit 11 against the penile skin of the subject, and the surrounding 60-Hz interference noise from the amplified standard pulse signal and the first amplified comparative pulse signal to obtain robust standard pulse signal and first robust comparative pulse signal. Finally, the first DC level-adjusting circuit 214 is operable so as to shift levels of the robust standard pulse signal and the first robust comparative pulse signal to a predetermined level that is larger than 0V to obtain the processed standard pulse signal and the first processed comparative pulse signal.

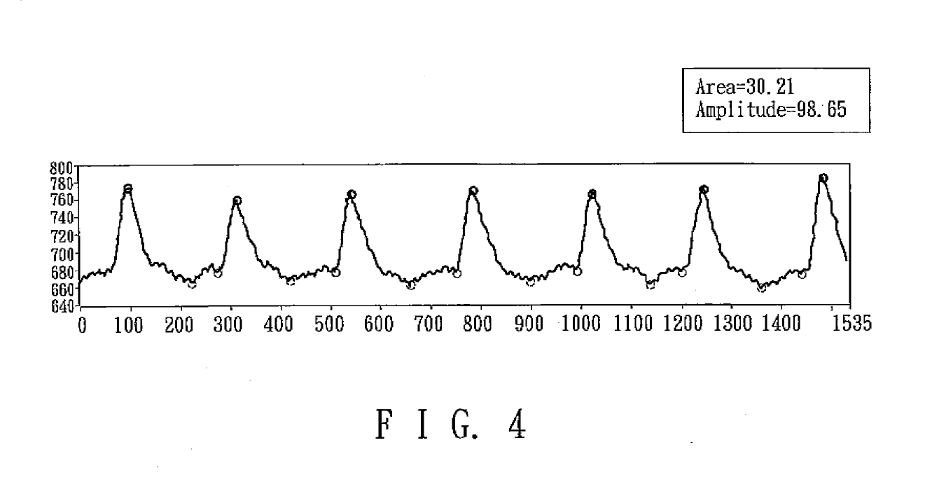

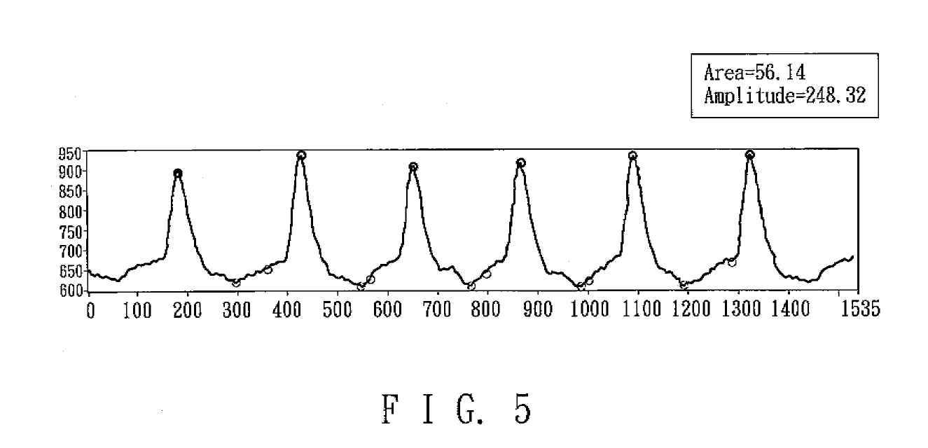

In step 35, the digitizing unit 22 is operable so as to digitize the processed standard pulse signal and the first processed comparative pulse signal to generate a digital standard pulse signal and a first digital comparative pulse signal as shown in FIGS. 4 and 5.

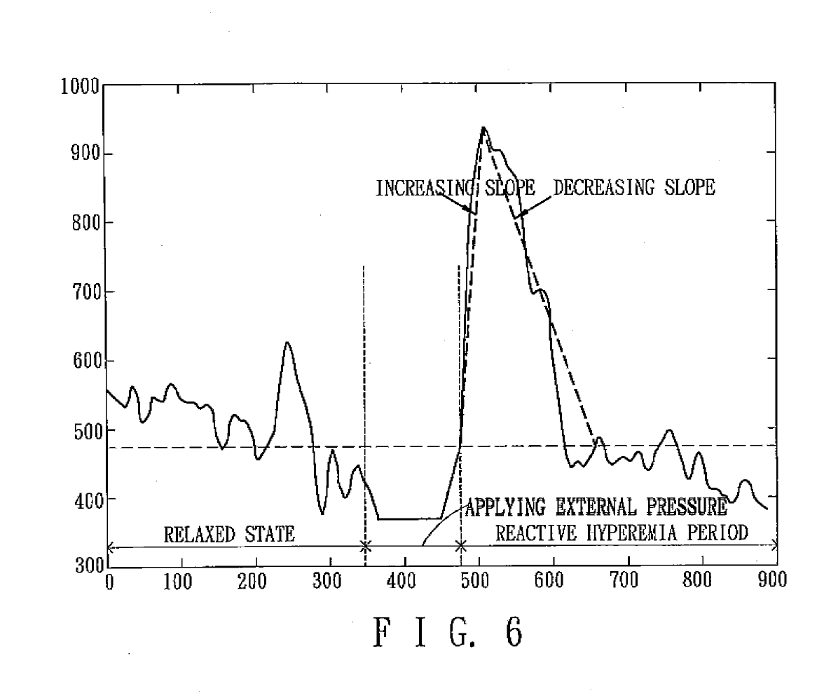

Finally, in step 36, according to signal parameters of the digital standard pulse signal and the first digital comparative pulse signal, the analyzing unit 23 is operable so as to obtain a vasodilatation index to serve as an erectile function index used for determining erectile function of the subject. For example, the vasodilatation index can be obtained according to area values or amplitude values with reference to the digital standard pulse signal and the first digital comparative pulse signal. Alternatively, the vasodilatation index can be obtained according to increasing slopes and decreasing slopes relative to waveforms of the digital standard pulse signal and the first digital comparative pulse signal under the reactive hyperemia period (see FIG. 6). The increasing and decreasing slopes of the waveforms are obtained by analyzing connection between peaks of the waveforms using Hilbert-Huang transformation.

In this embodiment, the analyzing unit 23 is operable to analyze peak values, valley values and starting points with reference to the digital standard pulse signal and the first digital comparative pulse signal, and then to perform the first algorithm 231 to compute the area values and the amplitude values (see FIGS. 4 and 5). Then, the analyzing unit 23 determines an average area value (A₁) and an average amplitude value (Amp₁) with reference to the digital standard pulse signal, and a maximum area value (A₂) and a maximum amplitude value (Amp₂) with reference to the first digital comparative pulse signal. In this embodiment, the average area value (A₁) and the average amplitude value (Amp₁) are calculated according to the area values and the amplitude values with reference to 20 cycles of the digital standard pulse signal before applying the external pressure to the penis of the subject. Finally, the analyzing unit 23 is operable to calculate a vasodilatation index with reference to the area values (DI-Area) based upon the following Equation (1), or a vasodilatation index with reference to the amplitude values (DI-Amplitude) based upon the following equation (2) The vasodilatation index (DI-Area, DI-Amplitude) can be used for determining function of penile endothelial cells of the subject, and serves as an erectile function index used for determining the erectile function of the subject.

$$\begin{matrix}DI \text{-} Area = \frac{{A}_{2}}{{A}_{1}} & ( 1 ) \\ DI \text{-} Amplitude = \frac{{Amp}_{2}}{{Amp}_{1}} & ( 2 )\end{matrix}$$

Concurrent with step 31, the electrocardiograph 14 is associated operatively with the electrode set 13 so as to measure the heartbeat of the subject, and obtain the electrocardiogram signal in step 41.

In step 42, the signal processing unit 21 is operable to perform filter processing upon the electrocardiogram signal to obtain a processed electrocardiogram signal. The instrumentation amplifier 215 has high common mode rejection ratio, and is operable so as to amplify the electrocardiogram signal and to remove a common-mode signal from the electrocardiogram signal in this step. Then, the noise-rejection filter 216 is operable so as to remove 60-Hz noise attributed to electric power source from the amplified electrocardiogram signal, and the band-pass filter 217 is operable to pass frequencies within a range from 0.98 Hz to 19.4 Hz to obtain a robust electrocardiogram signal. Finally, the second DC level-adjusting circuit 218 is operable so as to shift levels of the robust electrocardiogram signal to a predetermined level that is larger than 0V to obtain the processed electrocardiogram signal.

In step 43, the digitizing unit 22 is operable so as to digitize the processed electrocardiogram signal and generate a digital electrocardiogram signal. Finally, in step 44, according to a predetermined travel distance (L) and a time interval (T) between the digital electrocardiogram signal and the digital standard pulse signal obtained in step 35, the analyzing unit 23 is operable to perform the second algorithm 232 to calculate the PWV based upon the following Equation (3). The PWV can be used for determining a degree of penile atherosclerosis of the subject, and can serve as another erectile function index used for determining erectile function of the subject.

$$PWV = \frac{L}{T} \qquad ( 3 )$$

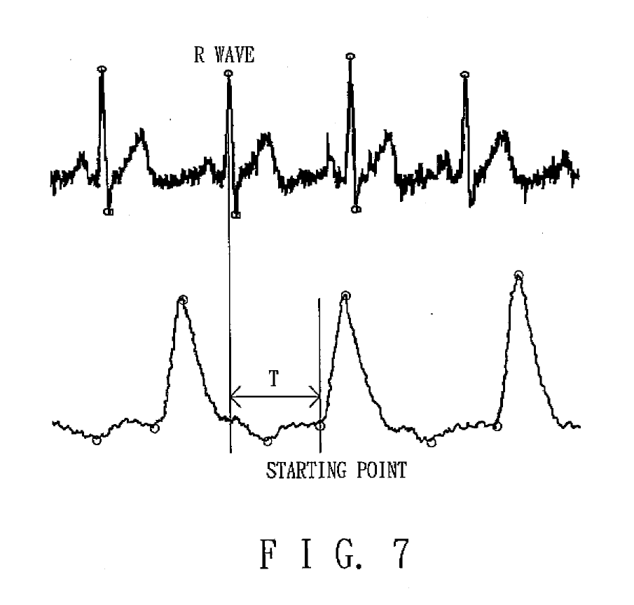

In Equation (3), T is a time interval between an R wave of the digital electrocardiogram signal and a corresponding one of starting points of the digital standard pulse signal as shown in FIG. 7, or a time interval between the R wave of the digital electrocardiogram signal and a corresponding one of peaks of the digital pulse signal. L is a distance from an incisura sternum of the subject to the penile artery of the subject as shown in FIG. 1.

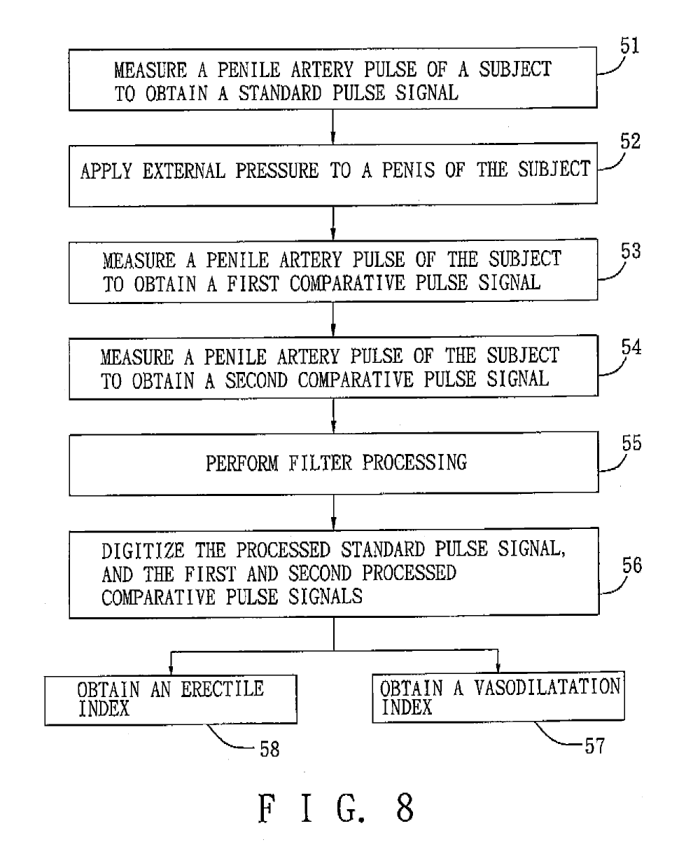

Referring to FIGS. 1 and 8, the second preferred embodiment of an erectile function index measuring and analyzing method performed using the system illustrated in FIG. 1 will now be described in the succeeding paragraphs.

In the second preferred embodiment, steps 51 to 53 are similar to steps 31 to 33 of the first preferred embodiment for obtaining a standard pulse signal and a first comparative pulse signal. In step 54, the pressure sensor 12 is associated operatively with the pressure applying unit 11 to output a second comparative pulse signal based upon a penile artery pulse of the subject that is measured while the penis of the subject is in an erect state. Preferably, the subject is under sexual stimulation for 2 minutes to result in the penis of the subject in the erect state, and then the pressure sensor 12 is operable to measure the penile artery pulse of the subject to obtain the second comparative pulse signal. It should be noted that, in other embodiments, step 54 may be implemented before step 53 or step 51 and is not limited to the disclosed sequence.

Subsequently, steps 55 and 56 of this embodiment are similar to steps 34 and 35 of the first preferred embodiment. In step 55, the signal processing unit 21 is operable so as to perform filter processing upon the standard pulse signal and the first and second comparative pulse signals to obtain a processed standard pulse signal and first and second processed comparative pulse signals. In step 56, the digitizing unit 22 is operable so as to digitize the processed standard pulse signal and the first and second processed comparative, pulse signals to generate a digital standard pulse signal and first and second digital comparative pulse signals.

In step 57, the analyzing unit 23 is operable so as to obtain a vasodilatation index according to signal parameters of the digital standard pulse signal and the first digital comparative pulse signal. Since the procedure for obtaining the vasodilatation index has been described in step 36 of the first preferred embodiment, details thereof will be omitted herein for the sake of brevity.

In step 58, according to signal parameters of the digital standard pulse signal and the second digital comparative pulse signal, the analyzing unit 23 is further operable to obtain an erectile index to serve as another erectile function index used for determining erectile function of the subject. For example, the erectile index can be obtained according to area values or amplitude values with reference to the digital standard pulse signal and the second digital comparative pulse signal. Alternatively, the erectile index can be obtained according to increasing slopes and decreasing slopes relative to waveforms of the digital standard pulse signal and the second digital comparative pulse signal under the reactive hyperemia period. The increasing and decreasing slopes of the waveforms are obtained by analyzing connection between peaks of the waveforms using Hilbert-Huang transformation.

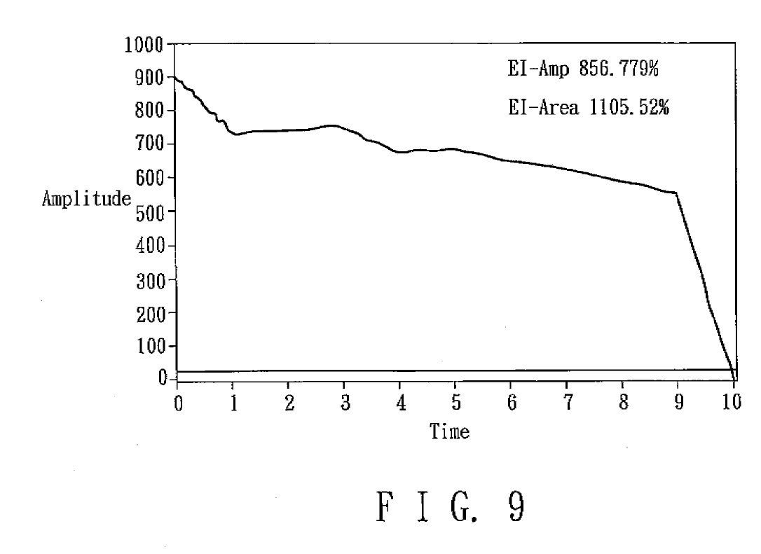

In this embodiment, the analyzing unit 23 is operable to analyze peak values, valley values and starting points with reference to the digital standard pulse signal and the second digital comparative pulse signal, and then to perform the third algorithm 233 to compute the area values and the amplitude values. Then, the analyzing unit 23 determines an average area value (A₁) and an average amplitude value (Amp₁) with reference to the digital standard pulse signal, and a maximum area value (A₃) and a maximum amplitude value (Amp₃) with reference to the second digital comparative pulse signal. In this embodiment, the average area value (A₁) and the average amplitude value (Amp₁) are calculated according to the area values and the amplitude values with reference to 20 cycles of the digital standard pulse signal before applying the external pressure to the penis of the subject. Finally, the analyzing unit 23 is operable to calculate an erectile index with reference to the area values (EI-Area) based upon the following Equation (4), or an erectile index with reference to the amplitude values (EI-Amplitude) based upon the following Equation (5). The erectile index (EI-Area, EI-Amplitude) is shown in FIG. 9, and can serve as another erectile function index used for determining the erectile function of the subject.

$$\begin{matrix}EI \text{-} Area = \frac{{A}_{3}}{{A}_{1}} & ( 4 ) \\ EI \text{-} Amplitude = \frac{{Amp}_{3}}{{Amp}_{1}} & ( 5 )\end{matrix}$$

The method of the second preferred embodiment may additionally includes steps 41 to 44 of the method of the first preferred embodiment.

In sum, the erectile function index measuring and analyzing system of the present invention is configured to obtain three erectile function indices including the vasodilatation index, the PWV and the erectile index. The erectile function indices can be used for determining the erectile function of the subject. Further, the erectile function index measuring and analyzing system can be used for long-term screening of the erectile function of the subject. By comparing the erectile function indices that are respectively obtained when the subject is in different conditions or at different time points, it can be determined whether, the erectile function of the subject is normal or whether a patient suffering from erectile dysfunction has recovered.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. An erectile function index measuring and analyzing method, comprising the steps of:
a) measuring a penile artery pulse of a subject to obtain a standard pulse signal while a penis of the subject is in a relaxed state;
b) obtaining at least one of a first comparative pulse signal based upon a penile artery pulse of the subject that is measured after releasing the penis of the subject from external pressure larger than a systolic pressure of the subject, and a second comparative pulse signal based upon a penile artery pulse of the subject that is measured while the penis of the subject is in an erect state;
c) processing the standard pulse signal and said at least one of the first and second comparative pulse signals to obtain a digital standard pulse signal and at least one of a first digital comparative pulse signal and a second digital comparative pulse signal, respectively; and
d) according to signal parameters of the digital standard pulse signal and said at least one of the first and second digital comparative pulse signals, obtaining at least one of a vasodilatation index with reference to the first digital comparative pulse signal and an erectile index with reference to the second digital comparative pulse signal to serve as an erectile function index used for determining erectile function of the subject.

2. The erectile function index measuring and analyzing method as claimed in claim 1, wherein, in step d), the vasodilatation index is obtained according to area values with reference to the digital standard pulse signal and the first digital comparative pulse signal.

3. The erectile function index measuring and analyzing method as claimed in claim 2, wherein the vasodilatation index is calculated as a quotient of a maximum area value with reference to the first digital comparative pulse signal divided by an average area value with reference to the digital standard pulse signal.

4. The erectile function index measuring and analyzing method as claimed in claim 1, wherein, in step d), the vasodilatation index is obtained according to amplitude values with reference to the digital standard pulse signal and the first digital comparative pulse signal.

5. The erectile function index measuring and analyzing method as claimed in claim 4, wherein the vasodilatation index is calculated as a quotient of a maximum amplitude value with reference to the first digital comparative pulse signal divided by an average amplitude value with reference to the digital standard pulse signal.

6. The erectile function index measuring and analyzing method as claimed in claim 1, wherein, in step d), the vasodilatation index is obtained according to increasing slopes and decreasing slopes relative to waveforms of the digital standard pulse signal and the first digital comparative pulse signal under a reactive hyperemia period, the increasing and decreasing slopes of the waveforms being obtained by analyzing connection between peaks of the waveforms using Hilbert-Huang transformation.

7. The erectile function index measuring and analyzing method as claimed in claim 1, wherein, in step d), the erectile index is obtained according to area values with reference to the digital standard pulse signal and the second digital comparative pulse signal.

8. The erectile function index measuring and analyzing method as claimed in claim 7, wherein the erectile index is calculated as a quotient of a maximum area value with reference to the second digital comparative pulse signal divided by an average area value with reference to the digital standard pulse signal.

9. The erectile function index measuring and analyzing method as claimed in claim 1, wherein, in step d), the erectile index is obtained according to amplitude values with reference to the digital standard pulse signal and the second digital comparative pulse signal.

10. The erectile function index measuring and analyzing method as claimed in claim 9, wherein the erectile index is calculated as a quotient of a maximum amplitude value with reference to the second digital comparative pulse signal divided by an average amplitude value with reference to the digital standard pulse signal.

11. The erectile function index measuring and analyzing method as claimed in claim 1, wherein, in step d), the erectile index is obtained according to increasing slopes and decreasing slopes relative to waveforms of the digital standard pulse signal and the second digital comparative pulse signal under a reactive hyperemia period, the increasing and decreasing slopes of the waveforms being obtained by analyzing connection between peaks of the waveforms.

12. The erectile function index measuring and analyzing method as claimed in claim 1, wherein:
in step a), heartbeat of the subject is further measured to obtain an electrocardiogram signal while the penis of the subject is in the relaxed state;
in step c), the electrocardiogram signal is processed to obtain a digital electrocardiogram signal; and
in step d), according to a predetermined travel distance and a time interval between the digital electrocardiogram signal and the digital standard pulse signal, a pulse wave velocity is obtained to serve as another erectile function index used for determining erectile function of the subject.

13. An erectile function index measuring and analyzing method, comprising the steps of:
i) while a penis of a subject is in a relaxed state, measuring heartbeat of the subject to obtain an electrocardiogram signal and measuring a penile artery pulse of the subject to obtain a standard pulse signal;
ii) processing the electrocardiogram signal and the standard pulse signal to obtain a digital electrocardiogram signal and a digital standard pulse signal; and
iii) according to a predetermined travel distance and a time interval between the digital electrocardiogram signal and the digital standard pulse signal, obtaining a pulse wave velocity to serve as an erectile function index used for determining erectile function of the subject.

14. The erectile function index measuring and analyzing method as claimed in claim 13, wherein, in step iii), the pulse wave velocity is calculated as a quotient of a distance from an incisura sternum of the subject to the penile artery of the subject divided by a time interval between an R wave of the digital electrocardiogram signal and a corresponding one of starting points of the digital standard pulse signal.

15. The erectile function index measuring and analyzing method as claimed in claim 13, wherein, in step iii), the pulse wave velocity is calculated as a quotient of a distance from an incisura sternum of the subject to the penile artery of the subject divided by a time interval between an R wave of the digital electrocardiogram signal and a corresponding one of peaks of the digital pulse signal.

16. An erectile function index measuring and analyzing system, comprising:
a measuring device that includes a pressure sensor configured to
output a standard pulse signal based upon a penile artery pulse of a subject that is measured while a penis of the subject is in a relaxed state, and
output at least one of a first comparative pulse signal based upon a penile artery pulse of the subject that is measured after releasing the penis of the subject from external pressure larger than a systolic pressure of the subject, and a second comparative pulse signal based upon a penile artery pulse of the subject that is measured while the penis of the subject is in an erect state; and
an analyzing device coupled to said measuring device, said analyzing device including
a signal processing unit configured to amplify and perform filter processing upon the standard pulse signal and said at least one of the first and second comparative pulse signals outputted by said pressure sensor of said measuring device to obtain a processed standard pulse signal and at least one of first and second processed comparative pulse signals,
a digitizing unit coupled to said signal processing unit and configured to digitize the processed standard pulse signal and said at least one of the first and second processed comparative pulse signals to obtain a digital standard pulse signal and at least one of a first digital comparative pulse signal and a second digital comparative pulse signal, and
an analyzing unit coupled to said digitizing unit and configured to analyze signal parameters of the digital standard pulse signal and said at least one of the first and second digital comparative pulse signals, and to obtain at least one of a vasodilatation index with reference to the first digital comparative pulse signal and an erectile index with reference to the second digital comparative pulse signal to serve as an erectile function index used for determining erectile function of the subject.

17. The erectile function index measuring and analyzing system as claimed in claim 16, wherein said analyzing unit of said analyzing device calculates the vasodilatation index as one of:
a quotient of a maximum area value with reference to the first digital comparative pulse signal divided by an average area value with reference to the digital standard pulse signal; and
a quotient of a maximum amplitude value with reference to the first digital comparative pulse signal divided by an average amplitude value with reference to the digital standard pulse signal.

18. The erectile function index measuring and analyzing system as claimed in claim 16, wherein said analyzing unit calculates the vasodilatation index according to increasing slopes and decreasing slopes relative to waveforms of the digital standard pulse signal and the first digital comparative pulse signal under a reactive hyperemia period, the increasing and decreasing slopes of the waveforms being obtained by analyzing connection between peaks of the waveforms using Hilbert-Huang transformation.

19. The erectile function index measuring and analyzing system as claimed in claim 16, wherein said pressure sensor of said measuring device is one of a clamping-type pressure sensor, a probe-type pressure sensor, and a sticker-pad-type pressure sensor.

20. The erectile function index measuring and analyzing system as claimed in claim 16, wherein:
said measuring device further includes an electrode set for application to a hand and a foot of the subject, and an electrocardiograph coupled to said electrode set;
said signal processing unit is further configured to amplify and perform filter processing upon the electrocardiogram signal to obtain a processed electrocardiogram signal;
said digitizing unit is further configured to digitize the processed electrocardiogram signal to obtain a digital electrocardiogram signal; and
said analyzing unit is further configured to analyze the digital standard pulse signal and the digital electrocardiogram signal, and to obtain a pulse wave velocity to serve as another erectile function index used for determining erectile function of the subject according to a predetermined travel distance and a time interval between the digital standard pulse signal and the digital electrocardiogram signal.

21. An erectile function index measuring and analyzing system, comprising:
a measuring device that includes a pressure sensor configured to output a standard pulse signal based upon a penile artery pulse of a subject that is measured while a penis of the subject is in a relaxed state, an electrode set for application to a hand and a foot of the subject, and an electrocardiograph coupled to said electrode set and configured to measure heartbeat of the subject and output an electrocardiogram signal concurrent with measuring of the penile artery pulse of the subject; and
an analyzing device coupled to said measuring device, said analyzing device including
a signal processing unit configured to amplify and perform filter processing upon the standard pulse signal and the electrocardiogram signal outputted by said measuring device to obtain a processed standard pulse signal and a processed electrocardiogram signal,
a digitizing unit coupled to said signal processing unit and configured to digitize the processed standard pulse signal and the processed electrocardiogram signal to obtain a digital standard pulse signal and a digital electrocardiogram signal, and
an analyzing unit coupled to said digitizing unit and configured to analyze the digital standard pulse signal and the digital electrocardiogram signal, and to obtain a pulse wave velocity to serve as an erectile function index used for determining erectile function of the subject according to a predetermined travel distance and a time interval between the digital standard pulse signal and the digital electrocardiogram signal.

22. The erectile function index measuring and analyzing method as claimed in claim 21, wherein said analyzing unit of said analyzing device calculates the pulse wave velocity as a quotient of a distance from an incisura sternum of the subject to the penile artery of the subject divided by a time interval between an R wave of the digital electrocardiogram signal and one of a corresponding starting point and a corresponding peak of the digital standard pulse signal.

23. The erectile function index measuring and analyzing system as claimed in claim 21, wherein said pressure sensor of said measuring device is one of a clamping-type pressure sensor, a probe-type pressure sensor, and a sticker-pad-type pressure sensor.
